(19) United States
(12) Patent Application Publication
(10) Pub. No.: US 2007/0283951 A1
(43) Pub. Date: Dec. 13, 2007

(54) Mask-nebulizer assembly

(76) Inventors: Marc Alan Burk, Murrieta, CA (US); Gary Roth, Murrieta, CA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/450,457

(22) Filed: Jun. 12, 2006

(51) Int. Cl. A61M 11/00

(52) U.S. Cl. 128/200.21; 128/200.14

(57) ABSTRACT

A respiratory therapy mask assembly comprises a mask configured to be secured on a patient's face, a substantially hollow pipe having an upper end secured in the mask inlet port, a lower end configured to receive a gas inlet pipe adapter, and an angled side pipe extension configured to receive a valved nebulizer adapter assembly, a gas inlet pipe adapter and integral gas inlet pipe secured in the lower end of the hollow pipe, and a valved nebulizer adapter assembly secured in the angled pipe extension and having a reciprocally movable spring-loaded valve biased to close, and a valve actuator configured to cooperate with a nebulizer for opening the spring-loaded valve.

### BACKGROUND OF THE INVENTION

The combination of a respiratory mask and a nebulizer is known in the art. Such an apparatus is shown and described, for example, in U.S. Pat. No. 5,586,551. Hudson RCI respiratory masks combined with nebulizers are commercially available from Teleflex Medical Products Division of Temecula, Calif. combining a high-output nebulizer secured on adult or pediatric masks. U.S. Pat. No. 5,701,886 also discloses a non-rebreather mask assembly for simultaneously delivering oxygen and an aerosolized medication to a patient.

### SUMMARY OF THE INVENTION

The apparatus described herein comprises a respiratory mask configured to be secured on a patient's face, a hollow elongated pipe connected to the mask for delivering oxygen to the mask having an angled side pipe extension and a valved nebulizer adapter secured on the angled side pipe extension. The valved nebulizer adapter comprises a reciprocally movable spring-loaded valve biased to a closed condition and configured to receive a nebulizer thereby forcing the spring-loaded valve to an open condition. Aerosol generated by a secured nebulizer is directed through the nebulizer adapter and into the elongated hollow pipe via the angled side pipe extension. The apparatus may be operated to simultaneously direct oxygen and an aerosol to the mask. The nebulizer may be disconnected from the apparatus without interfering with or otherwise interrupting the flow of oxygen delivered to a patient via the mask.

### BRIEF DESCRIPTION OF THE DRAWINGS

FIG. 1 illustrates one embodiment of the mask assembly showing the elongated hollow pipe for simultaneously delivering aerosol and oxygen to a mask, with portions of the pipe and mask partially cutaway to show component connections;

FIG. 2 illustrates another therapy mask embodiment with a nebulizer and gas reservoir bag; and

FIG. 3 is a sectional view of the elongated hollow pipe showing gas inlet pipe adapter and valved nebulizer adapter positioned in the hollow pipe.

### DETAILED DESCRIPTION OF PREFERRED EMBODIMENTS

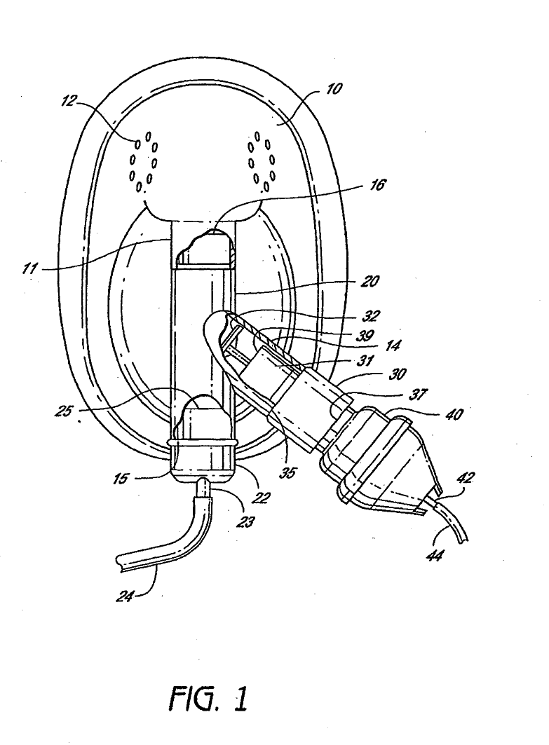

FIG. 1 illustrates a respiratory therapy mask assembly comprising a face mask 10, an elongated hollow pipe 20 secured on the mask via gas port 11, a nebulizer adapter assembly 30 and a nebulizer 40. The mask 10 is provided with exhalation ports of varying configurations on each side of the mask near a patient's nose and mouth. The exhalation ports allow patient breath to be vented to the atmosphere during expiration and some room air to be drawn into the mask during inspiration, depending on peak inspiratory flow. Such mask design is often referred to as a medium concentration mask. The mask illustrated is elongated to fit under the chin of a patient, although a shorter mask may also be used. An inlet port 11 on the mask is configured to receive the upper end 16 of elongated hollow pipe 20 visible through a cutaway portion of the inlet port 11. At the lower end of elongated hollow pipe 20 is secured gas inlet pipe adapter 22. The upper end 25 of inlet pipe adapter 22 is shown through the cutaway of the lower end of elongated hollow pipe 20. Pipe adapter 22 is provided with an integral gas inlet pipe 23 to which is secured a gas tubing 24 for directing oxygen or oxygen-containing respiratory gas from a source to the respiratory therapy mask. In the embodiment illustrated, gas inlet pipe adapter 22 is provided with a collar 15 for limiting the extent of insertion of the adapter pipe into elongated hollow pipe 20, although such a feature is optional. The installed pipe adapter 22 is in movable engagements at the end of the elongated hollow pipe 20. Thus, although the second pipe adapter is in gas sealed fit with the lower end of the pipe 20, e.g., force fit engagement, it is also rotatably secured so that the pipe adapter and integrated gas inlet pipe 23 can be conveniently rotated in a desired direction to avoid interference with angled side pipe 14, the nebulizer adapter assembly and an attached nebulizer and position the gas delivery tubing at desired angles without interfering with other equipment, tubing or components.

Angled side pipe extension 14 is also substantially hollow and provides a second inlet for directing fluid through the hollow pipe to the respiratory mask. The inlet port and angled side pipe extension 14 are configured to receive valved nebulizer adapter assembly 30. As illustrated, aerosol pipe 31 of the valved nebulizer adapter extends into angled pipe extension 14 to a position which allows aerosol generated by a nebulizer to be introduced into elongated hollow pipe 20. The valved nebulizer adapter assembly 30 may be provided with a shoulder 35 or other equivalent feature or component for limiting the insertion of the adapter assembly to a position which effectively secures the adapter assembly in the angled side pipe. The mating or engagement of the nebulizer adapter assembly and the side pipe may include force fit, threaded engagement or equivalent. For example, the nebulizer adapter assembly may be provided with a guide member or protuberance to be engaged in a slot or recess formed at the inlet port of the angled side pipe.

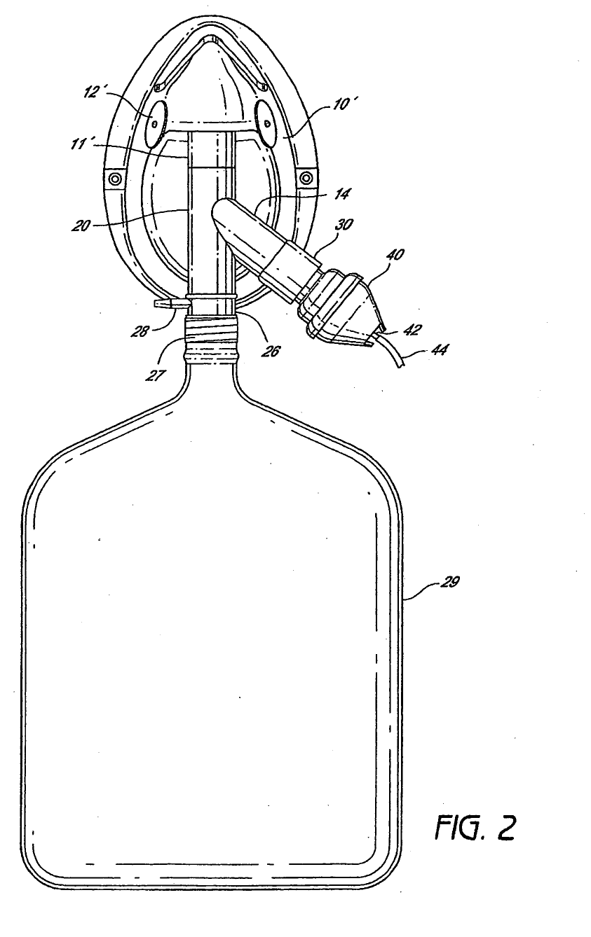

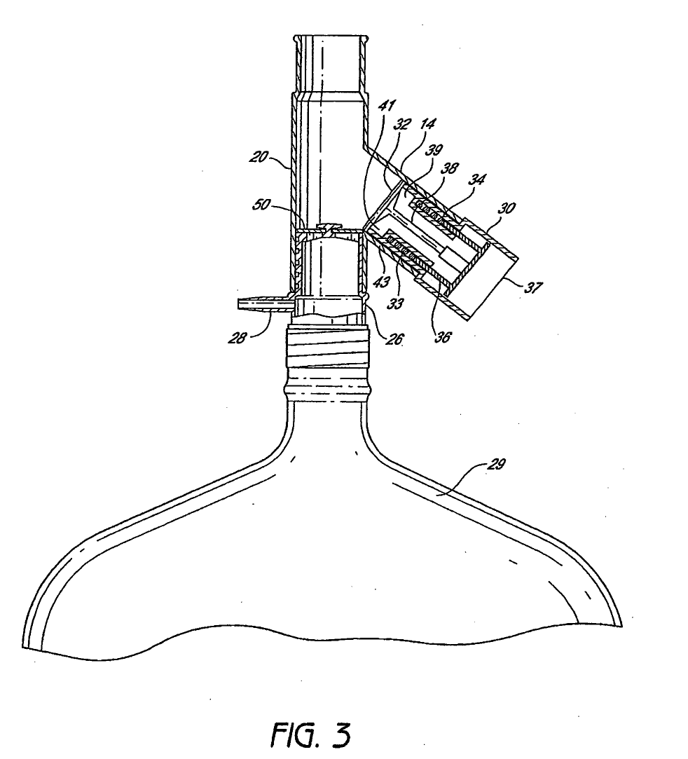

Referring also to FIG. 3, the valved nebulizer adapter assembly 30 includes an inlet port 37 configured to received an outlet pipe and port of a nebulizer and an aerosol outlet port 39. In the preferred embodiment illustrated, the valved nebulizer adapter assembly further comprises a valve 32 for closing aerosol outlet port 39. The valve is biased to the closed position by spring 34 which is housed in a chamber between inside wall 41 and outer wall 43. The normally closed valve 32 is opened by forcing the outlet pipe of a nebulizer against valve actuator 36 which compresses spring 34 and urges valve 32 to an open position via valve stem 38 structurally connected to valve actuator 36. A more complete description of a preferred valve nebulizer adapter assembly is disclosed in U.S. Pat. No. 6,725,858, the description of which is incorporated herein by reference in its entirety. Other valved nebulizer adapter assembly designs may also be used for example, such as described in U.S. Pat. No. 4,951,661. A nebulizer 40 inserted into aerosol inlet port 37 is also illustrated in FIGS. 1 and 2, the nebulizer provided with a gas supply pipe 42 and tubing 44. Any suitable nebulizer for delivering aerosol for patient respiratory therapy or other medical treatment may be used. Examples of such nebulizers are described in U.S. Pat. No. 4,588,129 and Pat. No. Re. 33,642, the descriptions of which are incorporated herein by reference.

FIG. 2 shows an alternative face mask and mask assembly incorporating a face mask 10′ which assembly is provided with a reservoir bag 29 secured adjacent to the lower end of elongated hollow pipe 20. Observing also FIG. 3, the assembly includes a one-way valve 50 positioned between the reservoir bag and the mask. In this embodiment, the assembly includes an adapter 26 having an integrally formed gas inlet pipe 28 extending outwardly along the length of the adapter. The upper end of adapter 26 is secured into the lower end of elongated hollow pipe 20, and at the lower end of adapter 26 is secured the reservoir bag 29. The upper end of adapter 26 is also provided with one-way valve 50 in the form of a flexible disc or wafer which allows a gas to be directed only one-way into elongated hollow pipe 20 from gas inlet pipe 28 and reservoir bag 29. Such a mask assembly design is referred to as a nonrebreathing mask. Alternatively, one-way valve 50 may be eliminated in which case the mask assembly operates as a partial rebreathing mask or high concentration mask. Either type of mask design may be used and is within the purview of the apparatus and assembly embodiments described and claimed herein. In the embodiment of FIG. 2, one-way valves 12′ are secured on the mask to prevent entrainment of atmospheric air during patient inspiration and ultimately provide for higher oxygen concentration delivered to the patient as compared to a mask in which one or more of the one-way mask valves are omitted. As also is shown in FIG. 2, reservoir bag 29 is typically secured to the lower end of adapter 26 with tape 27.

The respiratory therapy mask assembly described herein, regardless of the specific assembly configuration or embodiment, allows simultaneous respiratory gas and aerosol delivery to a patient, while also providing for selective removal of a nebulizer from the assembly without interrupting the respiratory gas flow and still maintaining the integrity of the gas delivery system by closure of the aerosol piping by simply detaching and removing the nebulizer. Such a feature does not require the user, patient or therapist to close the aerosol delivery limb of the assembly. Moreover, there is no additional requirement for introducing the aerosol other than attaching the nebulizer, thereby substantially simplifying operation and convenience of the apparatus.

What is claimed is:

1. A respiratory therapy mask assembly comprising:
a mask configured to be secured on a patient's face, said mask including one or more exhaust ports and an inlet port configured to receive a pipe;
a substantially hollow pipe having an upper end comprising an outlet port secured in the mask inlet port, a lower end comprising a first inlet port configured to receive a gas inlet pipe adapter, and an angled side pipe extension comprising a second inlet port configured to receive a valved nebulizer adapter assembly;
a gas inlet pipe adapter and integral gas inlet pipe secured in said first inlet port and configured to direct oxygen into said hollow pipe via said first inlet port; and
a valved nebulizer adapter assembly secured in said second inlet port and configured to direct aerosol from a nebulizer into said hollow pipe via said second inlet port and comprising an aerosol outlet end for being secured in said second inlet port, a reciprocally movable spring-loaded valve biased to close said aerosol outlet end, and a valve actuator configured to cooperate with a nebulizer for opening said valve.

2. A mask assembly of claim 1 including a gas reservoir bag secured to said gas inlet pipe adapter.

3. A mask assembly of claim 1 including a one-way valve secured on said gas inlet pipe adapter.

4. A mask assembly of claim 2 including a one-way valve secured on said gas inlet pipe adapter.

5. A mask assembly of claim 1 wherein said valved nebulizer adapter assembly comprises a spring and an elongated tubular passageway extending between said aerosol outlet end and an aerosol inlet end, wherein said valve actuator is located in said elongated tubular passageway, wherein said aerosol inlet end is configured to receive a nebulizer.

6. A mask assembly of claim 2 wherein said valved nebulizer adapter assembly comprises a spring and an elongated tubular passageway extending between said aerosol outlet end and an aerosol inlet end, wherein said valve actuator is located in said elongated tubular passageway, wherein said aerosol inlet end is configured to receive a nebulizer.

7. A mask assembly of claim 4 wherein said valved nebulizer adapter assembly comprises a spring and an elongated tubular passageway extending between said aerosol outlet end and an aerosol inlet end, wherein said valve actuator is located in said elongated tubular passageway, wherein said aerosol inlet end is configured to receive a nebulizer.

8. A mask assembly of claim 1 further comprising an aerosol generating nebulizer secured in valve actuator engagement in said valved nebulizer adapter assembly.

9. A mask assembly of claim 5 further comprising an aerosol generating nebulizer secured in valve actuator engagement in said valved nebulizer adapter assembly.
